(19) United States
(12) Patent Application Publication
(10) Pub. No.: US 2023/0172684 A1
(43) Pub. Date: Jun. 8, 2023

(54) INTELLIGENT ANALYTICS AND QUALITY ASSESSMENT FOR SURGICAL OPERATIONS AND PRACTICES

(71) Applicant: Genesis Medtech (USA) Inc., Saint Louis Park, MN (US)

(72) Inventors: Bin Zhao, Ellicott City, MD (US); Ning Li, Pittsburgh, PA (US); Shan Wan, Plymouth, MN (US)

(21) Appl. No.: 18/075,215

(22) Filed: Dec. 5, 2022

Related U.S. Application Data
(60) Provisional application No. 63/286,444, filed on Dec. 6, 2021.

(51) Int. Cl. A61B 90/00; G16H 20/40; G16H 70/20; G06V 20/40; G16H 30/40

(52) U.S. Cl. CPC A61B 90/37; G16H 20/40; G16H 70/20; G06V 20/41; G06V 20/46; G16H 30/40

(57) ABSTRACT

This disclosure describes a video-based surgery analytics and quality/skills assessment system. The system takes surgery video as input and generates rich analytics on the surgery. Multiple features may be extracted from the video that describe operation quality and surgeon skills, such as time spent on each step of the surgery, medical device movement trajectory characteristics, and adverse events occurrence such as excessive bleeding. Description of the surgery, related to its difficulty such as patient characteristics, may be also utilized to reflect surgery difficulty. Considering the various extracted features and the surgery description provided by surgeon, a machine learning based model may be trained to assess surgery quality and surgeon skills, by weighing and combining those factors. The system may solve 2 challenges in objective and reliable assessment: differing level of surgery difficulty caused by patient uniqueness and balancing multiple factors that affect quality and skills assessment.

### CROSS-REFERENCE TO RELATED APPLICATIONS

This application claims the benefit of priority to U.S. Provisional Application No. 63/286,444, filed Dec. 6, 2021, the entire disclosure of which is herein incorporated by reference in its entirety for all purposes.

### FIELD

This disclosure relates to medical operation analytics and quality/skills assessment, and more particularly, to video-based surgery analytics and quality/skills assessment based on multiple factors. The medical operations include a wide variety and broad range of operations, and they are not limited to the examples specifically mentioned herein.

### BACKGROUND

Timely feedback and assessment are paramount in surgeon training and growth. Current feedback mechanism relies on experienced surgeons reviewing surgeries and/or surgery videos to provide subjective assessment of procedure quality and surgeon skills. This is not only time-consuming, causing feedback and assessment to be sporadic, but also prone to inconsistency between assessors. Therefore, an automatic analytics and assessment system is desirable to provide objective quality assessment applicable to various procedures.

### SUMMARY

This disclosure is directed to medical operation analytics and quality/skills assessment. The analytics may be based on videos of medical operations like surgeries, and the quality/skills assessment may be based on multiple factors. Some method embodiments may include a method comprising: receiving a video that shows a medical operation performed on a patient; extracting a plurality of features from the video that shows the medical operation performed on the patient; receiving a description of the medical operation and the patient; generating an assessment of operation quality or skills in the medical operation, based on the description of the medical operation and the patient and based on the extracted plurality of features from the video; generating analytics on the medical operation of the video; and visualizing the analytics for user viewing, wherein the assessment of operation quality or skills in the medical operation and the analytics are shown for user viewing on a user interface.

Some system embodiments may include a system comprising: circuitry configured for: receiving a video that shows a medical operation performed on a patient, extracting a plurality of features from the video that shows the medical operation performed on the patient, receiving a description of the medical operation and the patient, generating an assessment of operation quality or skills in the medical operation, based on the description of the medical operation and the patient and based on the extracted plurality of features from the video, generating analytics on the medical operation of the video, and visualizing the analytics for user viewing; and storage for storing the generated assessment and the generated analytics, wherein the assessment of operation quality or skills in the medical operation and the analytics are shown for user viewing on a user interface.

Some non-transitory machine-readable medium embodiments may include a non-transitory machine-readable medium storing instructions, which when executed by one or more processors, cause the one or more processors to perform a method, the method comprising: receiving a video that shows a medical operation performed on a patient; extracting a plurality of features from the video that shows the medical operation performed on the patient; receiving a description of the medical operation and the patient; generating an assessment of operation quality or skills in the medical operation, based on the description of the medical operation and the patient and based on the extracted plurality of features from the video; generating analytics on the medical operation of the video; and visualizing the analytics for user viewing, wherein the assessment of operation quality or skills in the medical operation and the analytics are shown for user viewing on a user interface.

In some embodiments, the medical operation comprises a laparoscopic surgery. In some embodiments, the extracted plurality of features comprises time spent on each step of the medical operation, tracked movement of one or more medical instruments used in the medical operation, or occurrence of one or more adverse events during the medical operation. In some embodiments, the description of the medical operation and the patient indicates a level of difficulty or complexity of the medical operation. In some embodiments, the analytics comprise recognized phases and recognized medical devices from the video. In some embodiments, the assessment of operation quality or skills in the medical operation is generated via a machine learning model trained to assess the operation quality or skills based on a plurality of factors. In some embodiments, the machine learning model is trained based on one or more previous assessments of one or more previous medical operations, wherein the one or more previous assessments are used as label information for training the machine learning model, the machine learning model optimized to minimize discrepancy between the one or more previous assessments and the generated assessment.

### BRIEF DESCRIPTION OF THE DRAWINGS

FIG. 1 illustrates an exemplary embodiment of the video-based surgery analytics and quality/skills assessment system.

FIG. 2 is a workflow of intelligent analytics and quality assessment for surgical operations and practices.

FIG. 3 illustrates a screen layout of visualization of surgery phases/workflow.

FIG. 4 illustrates a screen layout of visualization of device usage information.

FIG. 5 illustrates a screen layout of visualization of analytics for a surgery type across different locations.

FIG. 6 illustrates a screen layout of visualization for a surgery in-progress.

FIG. 7 illustrates a screen layout of visualization for an upcoming surgery.

### DETAILED DESCRIPTION OF PREFERRED EMBODIMENTS

This disclosure is not limited to the particular systems, devices and methods described, as these may vary. The terminology used in the description is for the purpose of describing the particular versions or embodiments only, and is not intended to limit the scope. Various examples will now be described. This description provides specific details for a thorough understanding and enabling description of these examples. One skilled in the relevant art will understand, however, various examples may be practiced without many of these details. Likewise, one skilled in the relevant art will also understand that embodiments can include many other obvious features not described in detail herein. Additionally, some well-known structures or functions may not be shown or described in detail herein, so as to avoid unnecessarily obscuring the relevant description.

Typical laparoscopic surgery may take one to three hours. In the traditional proctoring model, the master or professor surgeon needs to stay close to the whole procedure, or review the surgery video afterward and give comments, which takes at least two to four hours. This feedback mechanism is time-consuming. Also, it is hard to get the master or professor surgeon the full length of time mentioned above. Thus, the master or professor surgeon can only provide feedback either at the point of being asked for input, or when he/she has a small time window, which means his/her feedback will be sporadic. Additionally, surgery in some sense has been taught like an art, and the assessment of artful skills are often subjective and can be inconsistent.

It is relatively straight forward to extract various features from the surgery video, relevant for quality or skills assessment. Example features include time spent on each surgery step, device movement trajectory characteristics, adverse events occurrence/frequency, etc. However, there are 2 main challenges in utilizing such extracted features for assessment:

1. Patient variance in severity of illness results in varying degrees of surgery difficulty, even for the same type of procedure. Such variance naturally affects some features such as surgery time, likelihood of adverse events such as excessive bleeding. A fair assessment should incorporate the varying difficulty and uniqueness of each procedure, even for same type of procedure. For example, two cholecystectomy procedures could have completely different levels of difficulty, due to patient characteristics such as deformity.

2. Multiple factors affect surgery quality and skills assessment, represented via the various features extracted from surgery video. How to effectively combine those factors to reach an objective and reliable assessment of operation quality and surgeon skills, is not well studied.

This disclosure describes a video-based surgery analytics and quality/skills assessment system. The system takes surgery video as input and generates rich analytics on the surgery. Multiple features are extracted from the surgery video that describe operation quality and surgeon skills, such as time spent on each step of the surgery workflow, medical device movement trajectory characteristics, and adverse events occurrence such as excessive bleeding. Description of the surgery, related to its difficulty such as patient characteristics, is also utilized to reflect surgery difficulty. Considering the various extracted features as well as the surgery description provided by surgeon, a machine learning based model is trained to assess surgery quality and surgeon skills, by properly weighing and combining those multiple factors.

The system solves 2 challenges in objective and reliable assessment of operation quality and surgeon skills: differing level of surgery difficulty caused by patient uniqueness and balancing multiple factors that affect quality and skills assessment.

Surgical videos may cover a wide variety of operations and are not limited to the specific examples recited herein. For example, surgical videos can either come from real laparoscopic surgeries or simulated environment/practices such as peg transfer exercise. The operations may include robotic and non-robotic operations, including robotic laparoscopic surgeries and non-robotic laparoscopic surgeries. Surgical videos may come from endoscopic surgeries and percutaneous procedures. The analytics and assessment can be used to compare against a benchmark, where a reference model is trained by an expert and serves as basis for the skills assessment; or against previous similar procedures performed by the same surgeon for longitudinal analysis of skills improvement. The benchmark may be generated for the system or by the system, and the system may generate evaluation scores for a subject surgeon, e.g., like alphabetic A/B/C/D/F scoring or numerical percentage scoring for students, by comparing with the benchmark. The reference model may be a model trained by exemplary video(s) of an expert surgeon(s). The analytics and/or assessment based on the analyzed video(s) can be used for patient education, peer review, proctoring, conference sharing, and fellowship teaching.

Surgery quality and surgeon skills assessment based on surgery video has been gaining popularity. The system described in this disclosure may have some or all of the following features:

1. Automatic analysis on video contents to generate rich analytics of the surgery video: The proposed system deploys video workflow analysis, object recognition and event recognition models to analyze the surgery video, to generate rich detection results, which are visualized to present insights about the surgery, beyond the raw frames.

2. Multiple features are extracted to serve as input signals for surgery quality and skills assessment. These features include surgeon provided description of the patient and surgery, as well as automatically detected features such as device movement trajectory, events, time spent in each step of the surgery workflow.

3. A machine learning model is trained to combine and properly weigh multiple features for final numerical assessment score. The model not only takes as inputs the various features extracted, but also considers the uniqueness and difficulty of the surgery, for an objective and fair assessment. Assessment scoring is not limited to numerical scores, but may include alphabetic scores, any metrics that differentiate performance level (such as novice/master/ . . . ), etc.

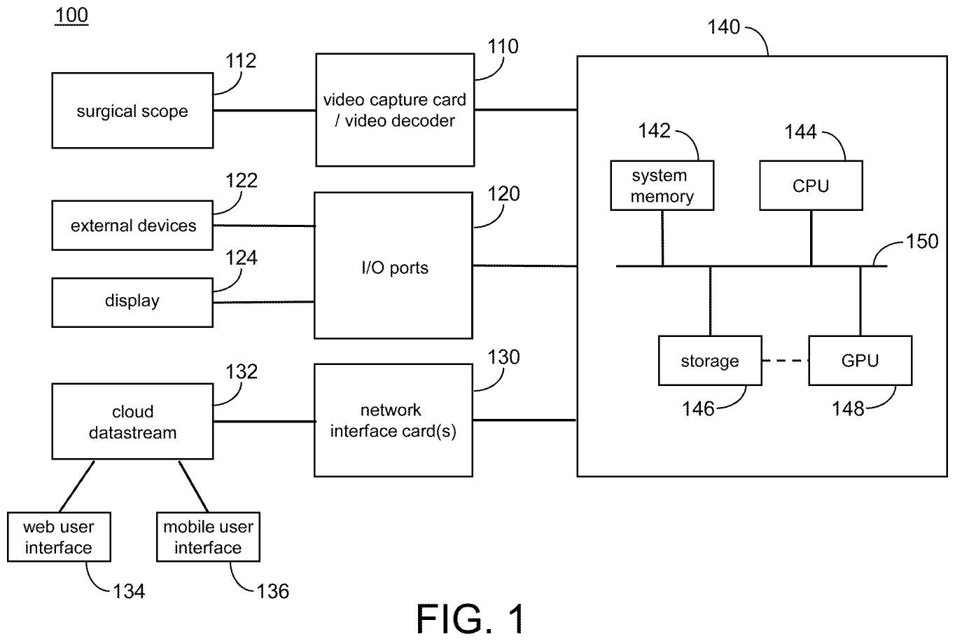

FIG. 1 illustrates an exemplary embodiment of the video-based surgery analytics and quality/skills assessment system 100. System 100 can provide surgery videos in one or more ways. System 100 can capture video image frames from surgical scope 112, e.g., by a video capture card/video decoder 110. Surgical scope 112 may be a laparoscope, endoscope, percutaneous scope, etc. that can provide a video feed across a video link, such as S-video, HDMI, etc. A camera(s) may be attached to, included inside as part of, or otherwise integrated with surgical scope 112, and may comprise a video camera that captures images, which may be sent from surgical scope 112. System 100 can receive video image frames at I/O ports 120 from external devices 122 (e.g., laptop, desktop computer, smartphone, data storage device, etc.) across a local data link, such as USB, Thunderbolt, etc. System 100 can receive video image frames at a network interface card(s) 130 from a cloud datastream 132 of a cloud network across a network data link, such as Ethernet, etc.

System 100 can perform analysis and assessment on video contents of the provided surgery videos at circuitry 140, which may be implemented as a motherboard. Circuitry 140 may include storage 146 (e.g., hard drive, solid-state drive, or other storage media) to store data, such as the surgery video(s), data for a machine learning model(s), user-provided data having description of patient and operation, data for a convolutional neural network(s), system software, etc. This storage 146 may include one or more storage medium devices that store data involved in the analysis and assessment on video contents of the provided surgery videos. Circuitry 140 may include circuitry 144, e.g., one or more CPUs or other kinds of processors, to execute software or firmware or other kinds of programs that cause circuitry 140 to perform the functions of circuitry 140. Circuitry 140 may include circuitry 148, e.g., one or more GPUs, to perform functions for machine learning. The CPU(s) and GPU(s) may perform functions involved in the analysis and assessment on video contents of the provided surgery videos. Throughout this disclosure, functions performed by GPU(s) 148 may also be performed by CPU(s) 144 or by GPU(s) 148 and CPU(s) 144 together. Circuity 140 may include system memory 142 (e.g., RAM, ROM, flash memory, or other memory media) to store data, such as data to operate circuitry 140, data for an operating system, data for system software, etc. Some or all of the components of circuitry 140 may be interconnected via one or more connections 150, like buses, cables, wires, traces, etc. In some embodiments, separate from connection(s) 150, GPU(s) 148 may be directly connected to storage 146, which may increase the speed of data transfer and/or reduce the latency of data transfer.

System 100 can provide the analysis and assessment of video contents of the provided surgery video to a user(s) in one or more ways. Circuitry 140 may connect to external devices 122 and display 124 via I/O ports 120 to provide the analysis and assessment to the user(s). External devices 122 may include user interface(s) (e.g., manual operators like button(s), rotary dial(s), switch(es), touch surface(s), touchscreen(s), stylus, trackpad(s), mouse, scroll wheel(s), keyboard key(s), etc.; audio equipment like microphone(s), speaker(s), etc.; visual equipment like camera(s), light(s), photosensor(s), etc.; any other conventional user interface equipment) to receive inputs from and/or provide outputs to the user(s), including outputs that convey the analysis and assessment. Display 124 can visualize the analysis and assessment. Display 124 may be a basic monitor or display that displays content of the analysis and assessment from circuitry 140 in a visual manner, or a more robust monitor or display system including circuitry that can perform some or all functionalities of circuitry 140 to perform the analysis and assessment, in addition to display components that can display content of the analysis and assessment in a visual manner. Display 124 may be a panel display that is housed or integrated with circuitry 140 or a separate display that can communicatively connect with circuitry 140, e.g., via a wired connection or a wireless connection. Display 124 may be housed or integrated with element(s) of external devices 122, such as in a monitor that includes a touchscreen, microphone, speakers, and a camera, to receive user inputs and to provide system outputs to a user. System 100 can similarly provide the analysis and assessment from circuitry 140 to user(s) at web user interface 134 and/or mobile user interface 135 via communications through network interface card(s) 130 and cloud datastream 132. Web user interface 134 and mobile user interface 135 may include similar user interface(s) and display(s) to receive inputs from and/or provide outputs to the user(s), including outputs that convey the analysis and assessment.

In some embodiments, circuitry 140 may include programs like an operating system, e.g., Linux, to run operations of circuitry 140. In some embodiments, circuitry 140 may include circuitry, e.g., FPGA or ASIC, or some combination of hardware circuitry and software to run operations of circuitry 140. Via some or all of the above components, circuitry 140 can receive surgery videos and perform analysis and assessment of video contents of the surgery videos.

The system may be implemented in various form factors and implementations. For example, the system can be deployed on a local machine, e.g., an independent surgery assistant system, integrated into a surgical scope (like laparoscope) product, or on a PC or workstation. As another example, the system can be deployed in an IT data server with on premise installation. As yet another example, the system will or may be a Software-as-a-Service (SaaS) product, deployed either in a secure public cloud or user's private could. User will or may be provided access to the system through a web user interface or mobile user interface. User can also provision access account to other members in their organization and define what contents are visible to each account.

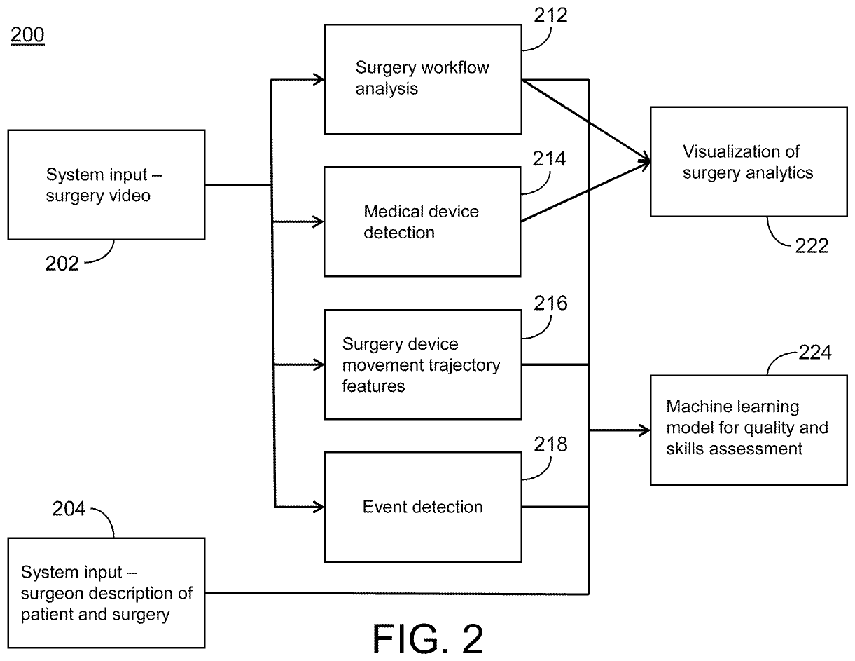

Assessment and analytics of surgery are complex and subject to multiple criteria. The system described in this disclosure will or may first allow users to upload their medical operation video, such as a surgery video (e.g., via cloud datastream 132 in FIG. 1), specify the procedure performed in the video, as well as provide description of the patient in terms of uniqueness that may cause complexity to the operation, and description of the operation. Then it will or may automatically analyze (e.g., via circuitry 140 in FIG. 1) the video to extract various features for objective assessment of the surgery. Besides assessment, the rich analytics generated by the system will or may also be shown to surgeon for self-reviewing (e.g., via display 124 in FIG. 1). Finally, these extracted features are or may be used as inputs to a machine learning model trained to assess surgery quality and surgeon skills. The system may work in following steps, and FIG. 2 gives details on the workflow 200.

1. Video Content Analysis and Visualizing Analytics

a. Surgery workflow analysis 212: Using pre-defined surgery phases/workflow for the specific procedure in the video, the system will or may automatically divide the surgery video 202 into segments corresponding to such defined phases. A machine learning model run on GPU(s) 148 in FIG. 1 may perform the auto-segmentation task. Starting and ending time stamps for each surgery phase is or may be automatically detected from the video 202, using video segmentation models trained by machine learning. A machine learning model run on GPU(s) 148 in FIG. 1 may perform the auto-detecting task.

b. Surgery device recognition 214: The system will or may also automatically recognize medical devices or tools used in each video frame. A machine learning model run on GPU(s) 148 in FIG. 1 may perform the device auto-recognition task.

c. The system will or may provide visualization 222 of the above phase recognition and device usage information on web/mobile user interface to give surgeon insights and analytics of the surgery.

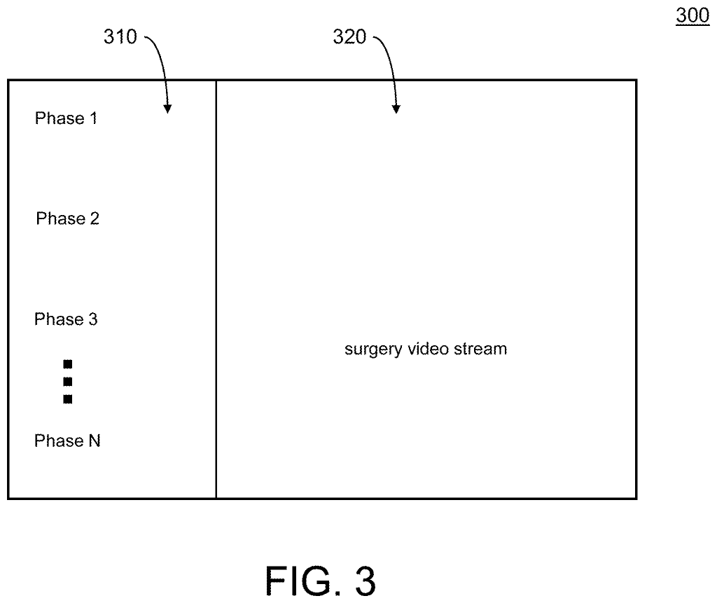

FIG. 3 illustrates a screen layout 300 of visualization of surgery phases/workflow. In example screen layout 300, the left side 310 may list or show the surgery phases 1, 2, 3, . . . , N corresponding to the auto-segmented surgery video, and the right side 320 may show the surgery video stream. The phases may be listed or shown with respective information about each respective phase, which may include insights and analytics of the surgery. Screen layout 300 may be displayed on display 124, on a display of web user interface 134, or on a display of mobile user interface 136 in FIG. 1.

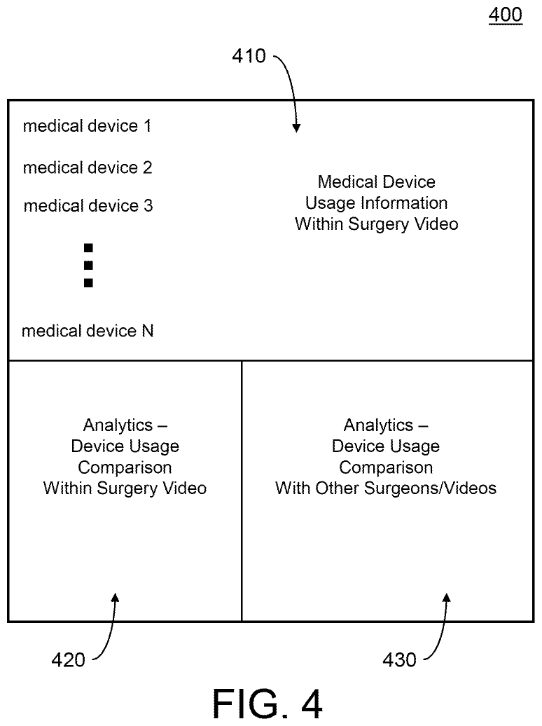

FIG. 4 illustrates a screen layout 400 of visualization of device usage information. In example screen layout 400, top panel 410 may visualize medical device usage information within a surgery video. The medical device usage information may include a listing or showing of the auto-detected medical devices or tools 1, 2, 3, . . . , N from the surgery video, total surgery time, total number of tools used, and usage statistics per tool. Bottom-left panel 420 may show analytics from within the surgery video, e.g., device usage comparison(s) within the single surgery video, such as most-used tool to least-used tool. Bottom-right panel 430 may show analytics across multiple surgery videos, e.g., device usage comparison(s) with other surgeons or other surgery videos, such as the tool usage times of the subject surgeon vs. tool usage surge times of other surgeon(s). Screen layout 400 may be displayed on display 124, on a display of web user interface 134, or on a display of mobile user interface 136 in FIG. 1. An artificial intelligence (AI) model run on GPU(s) 148 in FIG. 1 can generate visual suggestions for future tool use, e.g., for greater efficiency, which may be similar in purpose to suggestions from the human intuition of a master or professor surgeon.

2. Feature Extraction for Surgery Quality and Surgeon Skills Assessment

a. User provided description of patient and operation 204: It is common for surgeons to provide anonymous medical description of the patient, covering the diagnosis, any uniqueness of the medical condition that may affect complexity of the surgery, as well as the surgery plan and description of the operation. A user can provide such information to system 100, e.g., via external devices 122, web user interface 134, or mobile user interface 135, such as user interface(s) that can receive inputs from the user. Such text information will or may be stored (e.g., via storage 146 in FIG. 1) by the system and transformed into numerical features through natural language understanding (NLU) models such as sentence embedding (USE or BERT, etc.). Those numerical features can be used as a search index, and be used in intelligent search function(s). An NLU model run on GPU(s) 148 in FIG. 1 may perform the text-into-numerals transformation task.

For determining surgery complexity or difficulty, system 100 can receive an input according to a known grading/complexity/difficulty scale, such as the Parkland grading scale for cholecystitis. The Parkland grading scale (PGS) has severity grade levels 1-5 based on anatomy and inflammation, where grade 1 is a normal appearing gallbladder and grade 5 is a highly diseased gallbladder. A user can input to system 100 the PGS grade level of the patient's gallbladder, which system 100 can correlate to a certain level of complexity or difficulty for the corresponding surgery. Additionally or alternatively, machine learning model 224 automatically determine a PGS grade level or a corresponding level of complexity or difficulty for the corresponding surgery, based on input text information from a user.

b. Tracking surgery instrument movement 216: to assess surgeon skills, surgery instrument maneuver will or may be used as a crucial indicator. Besides recognizing the types of instruments being used in the video frames (e.g., as in surgery device recognition 214), the system will or may also locate the instrument and track its trajectory. Specifically, the system will or may identify the instrument tip and its spatial location within each video frame, to track the location, position, and trajectory of such devices. Features/cues extracted from device movement may include motion smoothness, acceleration, trajectory path length, occurrence/frequency of instruments outside of scope's view. A machine learning model run on GPU(s) 148 in FIG. 1 may perform the device auto-tracking task.

c. Event detection 218: the system will detect pre-defined events from surgery videos. These pre-defined events may be defined in advance by common medical practice or specific annotations from doctors or others. A machine learning model run on GPU(s) 148 in FIG. 1 may perform the pre-defined event auto-detection task. Important events in surgery include excessive bleeding, devices coming too close to important organ/tissue. For excessive bleeding, the system can train a convolutional neural network (CNN) to detect bleeding imagery in each frame or some frames of the surgery video. Output(s) of such a CNN trained to detect bleeding imagery can be used as input(s) to the final image classifier to assess quality and skills. For devices coming too close to important organ/tissue, determining what organ/tissue is important can be surgery-type dependent, e.g., different types of surgery may have a different or even unique definition of what organ/tissues is the important organ(s)/tissue(s). For example, in inguinal hernia repair surgery, the so-called “triangle of pain” is one such important tissue region to identify, as the surgeon may want devices to avoid coming too close to that region. The system can train a CNN to detect occurrence of such important organ/tissue in each frame or some frames, and detect whether certain surgical instrument(s), such as a surgical energy device, comes close to the tissue. Occurrence of such adverse events will or may be used as a factor in assessing procedure quality and surgeon skills.

d. From the surgery workflow analysis 212 results, time spent on each surgery step will or may be extracted and used as input features for quality and skills assessment.

3. Quality and Skills Assessment

a. The system will or may utilize 2 categories of information as inputs: surgeon's description of the patient and operation 204; automatically extracted features from the surgery video 202. The surgeon's description patient and operation 204 may be a description of the specific anonymous medical condition and/or surgery plan. For example, if there is any unique or characteristic aspect in a particular upcoming hernia repair surgery for a specific patient, a surgeon can make a description of such a unique or characteristic aspect(s), such as “this is a recurrent hernia that occurred near the site of a previous repair surgery.” Such information for the upcoming surgery may indicate information related to the difficulty of the upcoming surgery, e.g., a user input of a severity grade level, input text indicating a severity grade level or a corresponding level of complexity or difficulty for the upcoming surgery. From the surgery video 202, automatically extracted features may include outputs of trained CNN model(s) that detect features from raw video frames, including device movement trajectory from 216, events from 218, and/or time spent in each step of the surgery workflow from 212.

b. Machine learning model 224 training: the system will or may utilize expert knowledge in skills assessment by asking experienced surgeons to compare pairs of surgeries or provide numerical scores for individual surgeries. Here, the training may include obtaining ground truth labeling for quality and skills assessment that is accurate in the real world. The system can train a machine learning model to automatically assign score(s) to surgery videos to reflect assessment of quality and skills. For training input, surgeons or experts may provide such ground truth labeling as, e.g., “Surgery A is done better than Surgery B” or “On a scale of 1-5, Surgery C has a score of 3.” Such expert assessment will be used as label information for training the skills assessment model, which will be optimized to minimize discrepancy between system's assessment and expert's assessment. The system can use such ground truth labeling to train a model that can provide assessment scores that can match, or be similar to, the expert-provided ground truth labels. After such training, a machine learning model run on GPU(s) 148 in FIG. 1 may perform the auto-assessment task.

As reflected in FIG. 2, system 100 in FIG. 1 may combine different features (e.g., multiple input to 224) to provide assessment output from 224. Specifically, a machine learning model may be trained to use those multiple features as input factors, to generate assessment. Those features may be combined in that one or more of those features may be inputs to the machine learning model 224. After training, the machine learning model 224 may be used by system 100 to generate assessment on a new input surgery video 202, factoring in the surgeon's description of the patient and surgery 204 that may accompany surgery video 202. Trained on the multiple features, machine learning model 224 can weigh those multiple features to generate an assessment score on the new input surgery video. The assessment score may be presented to user(s) via display 124 and/or outputs of user interface(s) among external devices 122, via a display and/or outputs of web user interface 134, or via a display and/or outputs of web user interface 134.

Surgical analytics visualization can be broad in scope of content, beyond focusing on a single surgery video (e.g., in FIG. 3) or even comparing multiple surgery videos (e.g., in FIG. 4). The scope can encompass different surgery types across different locations, can be generated with multiple surgeries by different surgeon from different hospital, different country and using different technical method.

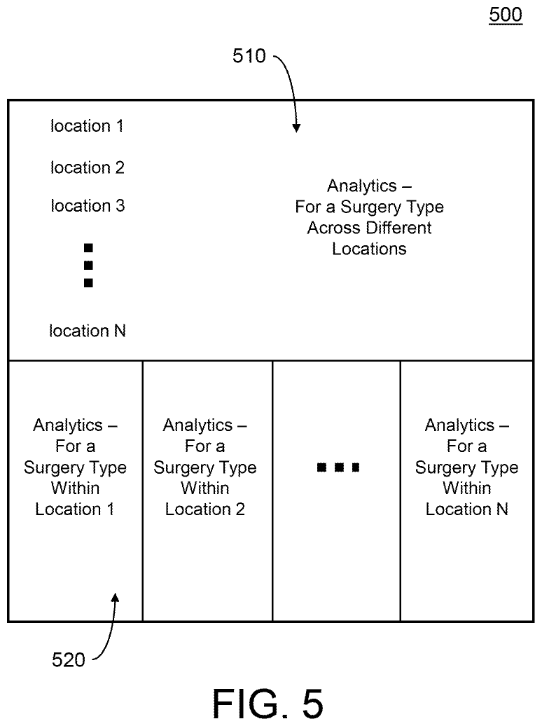

FIG. 5 illustrates a screen layout 500 of visualization of analytics for a surgery type across different locations. In example screen layout 500, top panel 510 may list or show different locations 1, 2, 3, . . . , N and analytics for a certain surgery type across those locations. The different locations may include different hospitals and countries. Each of the bottom panels 520 may show analytics for the surgery type, respectively for each of the different locations, such as which tools are used and for how long for that surgery type in that respective location. Accordingly, FIG. 5 can show how different locations use which tools and how long to perform the same surgery type. Screen layout 500 may be displayed on display 124, on a display of web user interface 134, or on a display of mobile user interface 136 in FIG. 1.

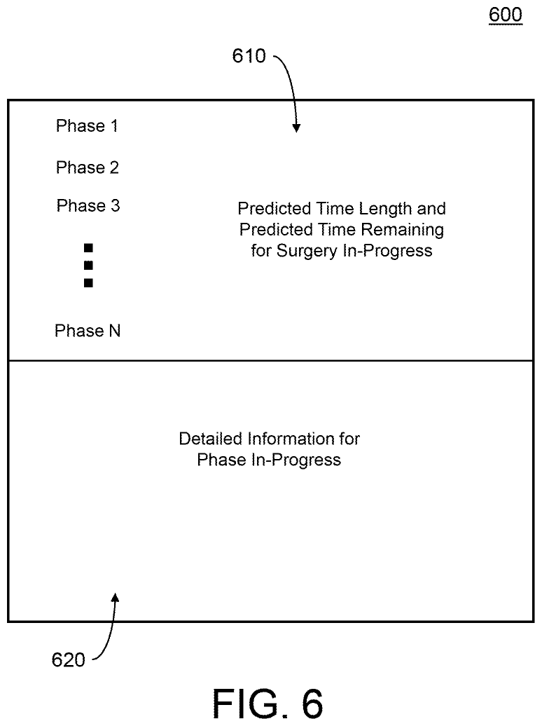

FIG. 6 illustrates a screen layout 600 of visualization for a surgery in-progress. In example screen layout 600, top panel 610 may list or show the surgery phases 1, 2, 3, . . . , N and predicted time information. For example, based on previous surgeries of the same type, system 100 can predict a time length and a time remaining for each phase of the surgery in-progress, as well as total time length and total time remaining for all phases. Bottom panel 620 may show detailed information for the phase in-progress, e.g., specific actions for the surgeon to do in that phase in-progress. Screen layout 600 may be displayed on display 124, on a display of web user interface 134, or on a display of mobile user interface 136 in FIG. 1.

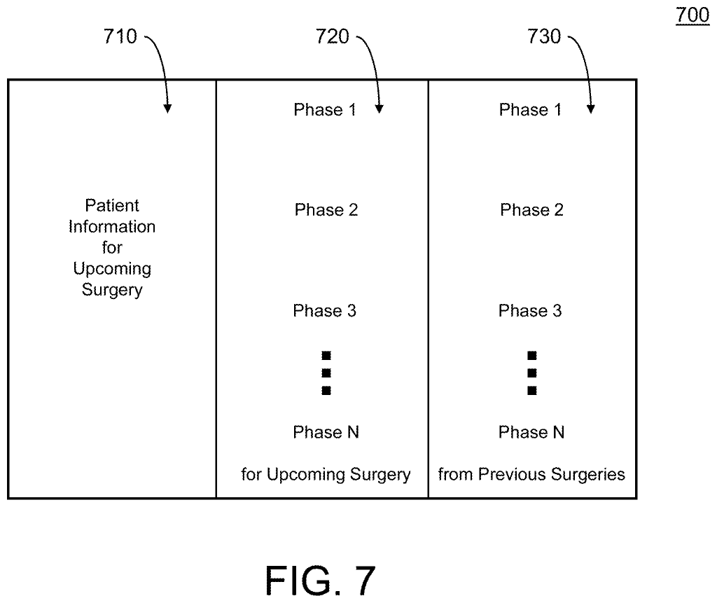

FIG. 7 illustrates a screen layout 700 of visualization for an upcoming surgery. In example screen layout 700, left panel 710 may present patient information for an upcoming surgery, e.g., general surgery plan and patent's background medical information. Center panel 720 may list or show the surgery phases 1, 2, 3, . . . , N for the upcoming surgery, e.g., main actions to do and predicted tools to use. Right panel 730 may list or show the surgery phases 1, 2, 3, . . . , N from previous surgeries of the same surgery type, e.g., highlights and procedure notes for each phase from the previous surgeries. Screen layout 700 may be displayed on display 124, on a display of web user interface 134, or on a display of mobile user interface 136 in FIG. 1.

Exemplary embodiments are shown and described in the present disclosure. It is to be understood that the embodiments are capable of use in various other combinations and environments and are capable of changes or modifications within the scope of the concepts as expressed herein. Some such variations may include using programs stored on non-transitory computer-readable media to enable computers and/or computer systems to carry our part or all of the method variations discussed above. Such variations are not to be regarded as departure from the spirit and scope of the invention, and all such modifications as would be obvious to one skilled in the art are intended to be included within the scope of the following claims.

What is claimed is:

1. A method comprising:
receiving a video that shows a medical operation performed on a patient;
extracting a plurality of features from the video that shows the medical operation performed on the patient;
receiving a description of the medical operation and the patient;
generating an assessment of operation quality or skills in the medical operation, based on the description of the medical operation and the patient and based on the extracted plurality of features from the video;
generating analytics on the medical operation of the video; and
visualizing the analytics for user viewing,
wherein the assessment of operation quality or skills in the medical operation and the analytics are shown for user viewing on a user interface.

2. The method of claim 1, wherein the medical operation comprises a laparoscopic surgery.

3. The method of claim 1, wherein the extracted plurality of features comprises time spent on each step of the medical operation, tracked movement of one or more medical instruments used in the medical operation, or occurrence of one or more adverse events during the medical operation.

4. The method of claim 1, wherein the description of the medical operation and the patient indicates a level of difficulty or complexity of the medical operation.

5. The method of claim 1, comprising:
recognizing in the video a plurality of phases of the medical operation; and
recognizing in the video one or more medical devices used in the medical operation,
wherein the analytics comprise the recognized phases and the recognized medical devices.

6. The method of claim 1, wherein the assessment of operation quality or skills in the medical operation is generated via a machine learning model trained to assess the operation quality or skills based on a plurality of factors.

7. The method of claim 6, wherein the machine learning model is trained based on one or more previous assessments of one or more previous medical operations, wherein the one or more previous assessments are used as label information for training the machine learning model, the machine learning model optimized to minimize discrepancy between the one or more previous assessments and the generated assessment.

8. A system comprising:
circuitry configured for:
receiving a video that shows a medical operation performed on a patient,
extracting a plurality of features from the video that shows the medical operation performed on the patient,
receiving a description of the medical operation and the patient,
generating an assessment of operation quality or skills in the medical operation, based on the description of the medical operation and the patient and based on the extracted plurality of features from the video,
generating analytics on the medical operation of the video, and
visualizing the analytics for user viewing; and
storage for storing the generated assessment and the generated analytics,
wherein the assessment of operation quality or skills in the medical operation and the analytics are shown for user viewing on a user interface.

9. The system of claim 8, wherein the medical operation comprises a laparoscopic surgery.

10. The system of claim 8, wherein the extracted plurality of features comprises time spent on each step of the medical operation, tracked movement of one or more medical instruments used in the medical operation, or occurrence of one or more adverse events during the medical operation.

11. The system of claim 8, wherein the description of the medical operation and the patient indicates a level of difficulty or complexity of the medical operation.

12. The system of claim 8, comprising:
recognizing in the video a plurality of phases of the medical operation; and
recognizing in the video one or more medical devices used in the medical operation,
wherein the analytics comprise the recognized phases and the recognized medical devices.

13. The system of claim 8, wherein the assessment of operation quality or skills in the medical operation is generated via a machine learning model trained to assess the operation quality or skills based on a plurality of factors.

14. The system of claim 13, wherein the machine learning model is trained based on one or more previous assessments of one or more previous medical operations, wherein the one or more previous assessments are used as label information for training the machine learning model, the machine learning model optimized to minimize discrepancy between the one or more previous assessments and the generated assessment.

15. A non-transitory machine-readable medium storing instructions, which when executed by one or more processors, cause the one or more processors and/or other one or more processors to perform a method, the method comprising:
receiving a video that shows a medical operation performed on a patient;
extracting a plurality of features from the video that shows the medical operation performed on the patient;
receiving a description of the medical operation and the patient;
generating an assessment of operation quality or skills in the medical operation, based on the description of the medical operation and the patient and based on the extracted plurality of features from the video;
generating analytics on the medical operation of the video; and
visualizing the analytics for user viewing,
wherein the assessment of operation quality or skills in the medical operation and the analytics are shown for user viewing on a user interface.

16. The non-transitory machine-readable medium of claim 15, wherein the extracted plurality of features comprises time spent on each step of the medical operation, tracked movement of one or more medical instruments used in the medical operation, or occurrence of one or more adverse events during the medical operation.

17. The non-transitory machine-readable medium of claim 15, wherein the description of the medical operation and the patient indicates a level of difficulty or complexity of the medical operation.

18. The non-transitory machine-readable medium of claim 15, the method comprising:
recognizing in the video a plurality of phases of the medical operation; and
recognizing in the video one or more medical devices used in the medical operation,
wherein the analytics comprise the recognized phases and the recognized medical devices.

19. The non-transitory machine-readable medium of claim 15, wherein the assessment of operation quality or skills in the medical operation is generated via a machine learning model trained to assess the operation quality or skills based on a plurality of factors.

20. The non-transitory machine-readable medium of claim 19, wherein the machine learning model is trained based on one or more previous assessments of one or more previous medical operations, wherein the one or more previous assessments are used as label information for training the machine learning model, the machine learning model optimized to minimize discrepancy between the one or more previous assessments and the generated assessment.
